(19) United States
(12) Patent Application Publication
(10) Pub. No.: US 2015/0140089 A1
(43) Pub. Date: May 21, 2015

(54) NOVEL FORMULATIONS OF NITROFURANS INCLUDING NIFURTIMOX WITH ENHANCED ACTIVITY WITH LOWER TOXICITY

(71) Applicant: MetronomX, Houston, TX (US)

(72) Inventors: Danong Chen, Houston, TX (US); Glenn Rice, Houston, TX (US)

(21) Appl. No.: 14/399,697

(22) PCT Filed: May 8, 2013
(86) PCT No.: PCT/US2013/040175

§ 371 Date: Nov. 7, 2014

Related U.S. Application Data
(60) Provisional application No. 61/644,252, filed on May 8, 2012.

(51) Int. Cl. A61K 31/541; A61K 9/50

(52) U.S. Cl. CPC A61K 31/541; A61K 9/5073
USPC 424/459; 514/227.8

(57) ABSTRACT

The present invention relates to a novel formulation of sustained-release Nifurtimox with enhanced activity while having low toxicity. The formulation can improve patient compliance by reducing the frequency of administering the drug.

### PRIOR RELATED APPLICATIONS

This invention claims priority to U.S. 61/644,252, filed on May 8, 2012 and incorporated by reference in its entirety herein.

### FEDERALLY SPONSORED RESEARCH STATEMENT

Not applicable.

### FIELD OF THE DISCLOSURE

The disclosure generally relates to novel formulations of nitrofurans, and more particularly to novel formulations including nifurtimox with enhanced activity with lower toxicity.

### BACKGROUND OF THE DISCLOSURE

Nifurtimox (3-methyl-4-(5′-nitrofurfurylidene-amino)-tetrahydro-4H-1,4-thiazine-1,1-dioxide), shown below as formula (1), is used to treat Chagas disease and has the potential of treating various types of cancers. Chagas disease is caused by the protozoan parasite Trypanosoma cruzi, which is widely distributed throughout the Americas, particularly in poor, rural areas of Mexico, Central America, and South America.

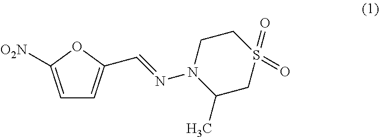 [SMILES: CC1CS(=O)(=O)CCN1/N=C/c1ccc([N+](=O)[O-])o1]

Marketed by Bayer as Lampit® tablets, Nifurtimox is administered three times a day with the duration of treatment ranging from 90 days (acute infection) to 120 days (chronic infection). This treatment is associated with significant toxicities, and patient compliance is also challenging. The pharmacological activities of Nifurtimox are related to its blood level; the active compound has a relatively short time to peak (1-2 h) and a short half-life (2.95±1.19 h), and the toxicities are likely related to the peak effects. Currently available Lampit® tablet (Nifurtimox) dosing regimen is T.I.D. The objective of this invention is to develop a multi-particulate sustained release (SR) capsule formulation with optimal absorption over the dosing interval with a daily frequency of dosing of QD or possibly BID. To do so, we employ an extrusion and spheronization process.

Nifurtimox is also believed to have the potency to treat cancers. Non-limiting examples of cancers treatable by Nifurtimox include: neuroblastoma, medulloblastoma, peripheral malignant nerve sheath tumor, ependymoma, chraniopharyngioma, astrocytoma, meningioma, germinoma, glioma, mixed glioma, choroid plexus tumor, oligodendroglioma, peripheral neuroectodennal tumor, primitive neuroectodermal tumor (PNET), CNS lymphoma, pituitary adenoma, Schwannoma, basal cell carcinoma, biliary tract cancer, bladder cancer, bone cancer, brain and CNS cancer, breast cancer, cervical cancer, choriocarcinoma, colon and rectum cancer, connective tissue cancer, cancer of the digestive system, endometrial cancer, esophageal cancer, eye cancer, fibroma, cancer of the head and neck, gastric cancer, intra-epithelial neoplasm, kidney cancer, larynx cancer, leukemia including acute myeloid leukemia, acute lymphoid leukemia, chronic myeloid leukemia, chronic lymphoid leukemia, liver cancer, small cell lung cancer, non-small cell lung cancer, lymphoma including Hodgkin's and Non-Hodgkin's lymphoma, melanoma, oral cavity cancer, ovarian cancer, pancreatic cancer, prostate cancer, retinoblastoma, rhabdomyosarcoma, rectal cancer, renal cancer, cancer of the respiratory system, sarcoma, skin cancer, stomach cancer, testicular cancer, thyroid cancer, uterine cancer, cancer of the urinary system, carcinomas or sarcomas.

Multi-particulate dosage forms have several advantages in comparison to single-unit dosage forms like tablets. In a multi-particulate form, the dosage of the drug substance is divided into sub-units consisting of thousands of spherical particles. Although the manufacture and design of these particles is more complex than that of tablets, multi-particulate dosage forms offer options and advantages to provide unique product characteristics, such as specific drug release patterns. Unlike non-disintegrating, monolithic, single-unit forms, which retain their structure in the digestive tract, multi-particulate preparations consist of numerous sub-units that disperse after administration. Because each sub-unit acts as an individual modified release entity, the multiple-unit approach offers certain advantages for a modified release dosage form, namely, a stable plasma profile, little risk of local side effects, reduced dependency on the nutrition state, reduced risk of dose dumping, and low intra- and inter-individual variability. An optimized pharmacokinetic profile with good patient compliance can be achieved with multi-particulate dosage forms.

The current Nifurtimox dosage regimen, toxicity, pharmacokinetic parameters (short time to peak and short half life) and the advantages of multi-particulate dosage forms (reduced risk of toxicity, good patient compliance, and stable plasma profile) make Nifurtimox a potential candidate for development of a Sustained Release multi-particulate dosage form.

### SUMMARY OF THE DISCLOSURE

The present invention relates to a novel multi-particulate dosage form for sustained-released Nifurtimox formulations. With different compositions of the formulation, the water-insoluble Nifurtimox can be continuously released for 24 hours or longer, thus improving patient's compliance with once-a-day regimen instead of T.I.D.

The sustained-released formulation of Nifurtimox comprises a therapeutically effective amount of Nifurtimox, a water-swellable hydrophilic polymer, and a binder, such that the formulation can continuously release Nifurtimox for up to 24 hours.

As used herein, “binder” means a compound that holds particulate ingredients that may be included in a tablet and/or particulates and the other ingredients in a tablet together. Binders are classified according to their application: solution binders are dissolved in a solvent (for example water or alcohol can be used in wet granulation processes). Examples of solution binders include gelatin, cellulose, cellulose derivatives, polyvinylpyrrolidone, starch, sucrose and polyethylene glycol. Dry binders are added to the powder blend, either after a wet granulation step, or as part of a direct powder compression (DC) formula. Examples include cellulose, methylcellulose, polyvinylpyrrolidone and polyethylene glycol. Binders are well known in the art of preparing pharmaceuticals, and additional non-limiting examples include acacia, alginic acid, carboxymethylcellulose sodium, microcrystalline cellulose, citric acid, dextrin, ethylcellulose, glucose, guar gum, hydroxypropyl methylcellulose, polyethylene oxide, povidone, pregelatinised starch, syrup, lactose, polyvinylpyrrolidone/vinyl acetate copolymer, and the like. Microcrystalline cellulose is the preferred binder. Microcrystalline cellulose is commercially available as Avicel® PH (pharmaceutical grade) from FMC Corporation, Philadelphia, Pa., particularly Avicel® PH 101, PH 102, PH 103, PH 112, PH 200, PH 301, PH 302 and Ceolus. In some cases, pressure-responsive excipient may be used in the formulation.

As used herein, “water-swellable hydrophilic polymer” refers to polymers that increase its volume upon contacting water due to crosslinking, thereby altering the drug release profile inside the GI tract. Suitable examples of water-swellable hydrophilic polymers include celluloses, such as carboxymethyl cellulose sodium, carboxymethyl cellulose, hydroxypropyl methylcellulose or hypromellose (“HPMC”), methylcellulose, hydroxymethylcellulose, hydroxyethylcellulose, hydroxy propyl cellulose (HPC), polyvinyl pyrrolidones, high-molecular weight polyvinylalcohols, and copolymers thereof; gums, such as natural gum, agar, agrose, sodium alginate, carrageenan, fucoidan, furcellaran, laminaran, hypnea, eucheums, gum Arabic, gum ghatti, gum karaya, gum tragacanth and locust bean gum; hydrophilic colloids, such as alginates, carbopols and polyacrylamides; other substances, such as arbinoglactan, pectin, amylopectin, gelatin, N-vinyl lactams and polysaccharides.

Hydroxypropyl methylcellulose or “HPMC” or “hypromellose” is partly O-methylated and 0-(2-hydroxypropylated) cellulose. It is available in various grades, such as K4M, K15M, K35M, K100M, K200M, K100LV, E3, E5, E6, E15 and E50 varying in viscosity and extent of substitution. In oral solid dosage forms, it is primarily used as a tablet binder, film-former, and as a matrix for use in extended-release tablet formulations.

As used herein, “enteric polymer” means a polymer, whose solubility is dependent on the pH in such a manner that it generally prevents the release of the drug in the stomach, but permits the release of the drug in the gastrointestinal tract at some stage after the particles have passed from the stomach.

As used herein, “pharmaceutically acceptable” means that the modified noun is appropriate for use in a pharmaceutical product; that is the pharmaceutically acceptable material is relatively safe and/or non-toxic, though not necessarily providing a separable therapeutic benefit by itself.

As used herein, “pharmaceutically effective amount” means an amount of a pharmaceutical active compound, or a combination of compounds, when administered alone or in combination, to treat, prevent, or reduce the risk of a disease state or condition. The term also refers to an amount of a pharmaceutical composition containing an active compound or combination of compounds. For example, a pharmaceutically effective amount refers to an amount of the pharmaceutical active present in a pharmaceutical composition or formulation of the present invention or on a medical device containing a composition or formulation of the present invention given to a recipient patient or subject sufficient to elicit biological activity, for example, activity against a known disease.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

### BRIEF DESCRIPTION OF THE DRAWINGS

FIG. 1 shows the microscopic images of the microparticulate beads of different Nifurtimox sustained release capsule formulations of this invention.

FIG. 2 is the bead size distribution curves of different Nifurtimox sustained release formulations of this invention.

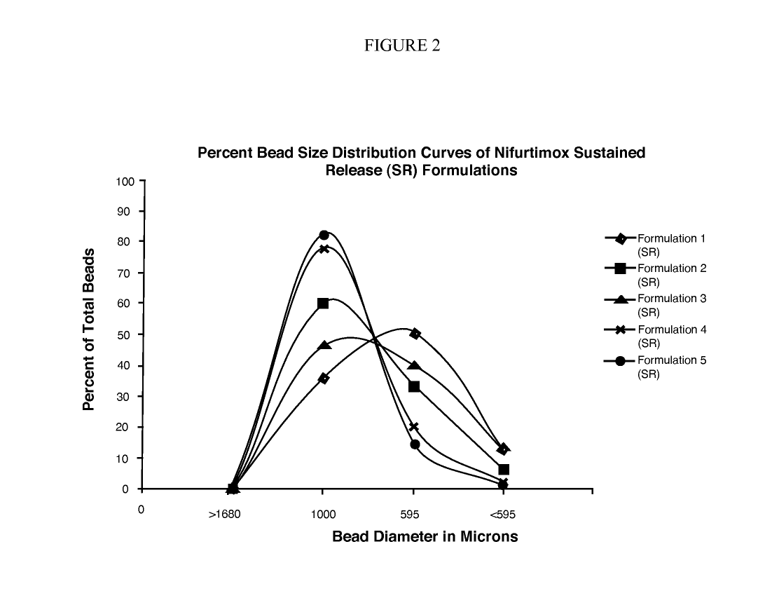

FIG. 3 shows the dissolution profiles of different Nifurtimox sustained release capsule formulations in 0.1N HCl with 5% HCl.

FIG. 4 shows the dissolution profiles of different Nifurtimox sustained release capsule formulations in 2% w/v SDS in deionized water.

### DETAILED DESCRIPTION

The disclosure provides novel Nifurtimox sustained release capsule formulations that are capable of continuously releasing Nifurtimox for 24 hours or more. The formulations comprise a therapeutically effective amount of Nifurtimox, a water-swellable hydrophilic polymer, and a binder. The formulation releases less than 50% of Nifurtimox after 8 hours, and releases more than 70% of Nifurtimox after 24 hours, thus showing sustained-release characteristics.

In one aspect of this invention, a formulation for controlled release of Nifurtimox is provided, wherein the formulation comprises a therapeutically effective amount of Nifurtimox, a water-swellable hydrophilic polymer, and a binder, wherein the formulation can continuously release Nifurtimox for at least 24 hours.

In another aspect of this invention, a method for treating a patient having Chagas disease or cancer is provided. The method comprises the steps of: administering, once daily, to the patient a sustained release capsule formulation of Nifurtimox, wherein the sustained release capsule formulation of Nifurtimox comprises a therapeutically effective amount of Nifurtimox, a water-swellable hydrophilic polymer, and a binder. The sustained release capsule formulation of Nifurtimox continuously releases Nifurtimox for at least 24 hours.

In one embodiment, the formulation is in multi-particulate form enclosed in a capsule. In another embodiment, the capsule comprises enteric materials. In yet another embodiment, the particulates in the multi-particulate formulation is further coated by an enteric material.

In one embodiment, the formulation comprises 10 to 40% w/w on dry solids basis of Nifurtimox. In a preferred embodiment, the formulation comprises 15 to 35% w/w on dry solids basis of Nifurtimox, and in a more preferred embodiment the formulation comprises 20 to 30% w/w on dry solids basis of Nifurtimox.

The water-swellable hydrophilic polymers that can be used in this formulation include but not limited to celluloses, such as carboxymethyl cellulose sodium, carboxymethyl cellulose, hydroxypropyl methylcellulose or hypromellose (“HPMC”), methylcellulose, hydroxymethylcellulose, hydroxyethylcellulose, hydroxy propyl cellulose (HPC), polyvinyl pyrrolidones, high-molecular weight polyvinylalcohols, and copolymers thereof; gums, such as natural gum, agar, agrose, sodium alginate, carrageenan, fucoidan, furcellaran, laminaran, hypnea, eucheums, gum Arabic, gum ghatti, gum karaya, gum tragacanth and locust bean gum; hydrophilic colloids, such as alginates, carbopols and polyacrylamides; other substances, such as arbinoglactan, pectin, amylopectin, gelatin, N-vinyl lactams and polysaccharides. Preferred water swellable hydrophilic polymer is Eudragit®RS PO, Eudragit®RL PO manufactured by Evonik Industries, Methocel K 100 Premium LV CR by Colorcon, Inc., or combinations thereof.

In one embodiment, the formulation comprises 1 to 40% w/w on dry solids basis of water-swellable hydrophilic polymer. In another embodiment, the formulation comprises 20 to 40% w/w on dry solids basis of water-swellable hydrophilic polymer. In yet another embodiment, the formulation comprises 1 to 10% w/w on dry solids basis of water-swellable hydrophilic polymer.

The binders that can be used in this formulation include but not limited to gelatin, cellulose, cellulose derivatives, polyvinylpyrrolidone, starch, sucrose and polyethylene glycol, cellulose, methylcellulose, polyvinylpyrrolidone and polyethylene glycol, acacia, alginic acid, carboxymethylcellulose sodium, microcrystalline cellulose, citric acid, dextrin, ethylcellulose, glucose, guar gum, hydroxypropyl methylcellulose, polyethylene oxide, povidone, pregelatinised starch, syrup, lactose, polyvinylpyrrolidone/vinyl acetate copolymer, and the like. In a preferred embodiment, microcrystalline cellulose is used as the binder. In a preferred embodiment, Avicel® PH 101 or PH 102, or combinations thereof, made by FMC Corporation, Philadelphia, Pa., is used as the binder.

In one embodiment, the formulation comprises 20 to 60% w/w on dry solids basis of binder. In another embodiment, the formulation comprises 25 to 55% w/w on dry solids basis of binder. In another embodiment, the formulation comprises 30 to 50% w/w on dry solids basis of binder.

The materials that can be employed in making drug-containing particles or microparticulates are any of those commonly used in pharmaceutics and should be selected on the basis of compatibility with the active drug and the physicochemical properties of the pellets.

Acrylic polymers are widely used as tablet coatings and as retardants of drug release in sustained released formulations. The commonly used acrylic polymers are high permeable Eudragit® RL and low permeable Eudragit® RS, both of which are neutral copolymers of poly (ethylacrylate, methyl methacrylate) and trimethyl aminoethyl methacrylate chloride, and are insoluble in water and digestive juices; but they swell and are permeable, which means that drugs embedded in their matrices can be released by diffusion. Therefore, the permeability of drug through Eudragit RS and/or RL is independent of the pH of the digestive tract. The degree of permeability depends on the relative proportion of quaternary ammonium groups in Eudragit®. The proportion of functional quaternary ammonium groups in Eudragit® RL and Eudragit® RS is 10 and 5%, respectively. Eudragit® RL PO and RS PO are fine, white powders with a slight aminelike odor. They are characteristically the same polymers as Eudragit® RS and RL.

In some embodiment, the extruded and spheronized Nifurtimox beads or particulates can further be coated to achieve different release profile. For example, the beads can be coated by enteric polymers in addition to the sustained release formulation.

Enteric polymers that may be used in the oral pharmaceutical formulation include but are not limited to: hydroxypropyl methylcellulose acetate succinate (HPMCAS), hydroxypropyl methylcellulose phthalate (HPMCP), polyvinyl acetate phthalate, cellulose acetate phthalate, cellulose acetate trimellitate, shellac, zein, polymethacrylates containing carboxyl groups, amylose acetate phthalate, styrene maleic acid copolymer, and cellulose acetate succinate. Examples of commercially available enteric material are available under the trade names EUDRAGIT® L 100 (methyl methacrylate/methacrylic acid copolymers wherein the ratio of free carboxyl groups to esters is about 1:1), EUDRAGIT® S 100 (methacrylic acid/methyl methacrylate copolymer with a 1:2 ratio of MA to MMA) or EUDRAGIT® L (methacrylic acid/methyl methacrylate copolymer with a 1:1 ratio of MA to MMA). Aqueous colloidal polymer dispersions or re-dispersions can be also applied, e.g. EUDRAGIT® L 30D-55 (methacrylic acid/ethyl acrylate copolymer), EUDRAGIT® L100-55 (ethyl acrylate, methacrylic acid copolymer), EUDRAGIT® preparation 4110D (methacrylic acid/methyl acrylate/methyl methacrylate copolymers wherein the ratio of methacrylic acid, methyl acrylate and methyl methacrylate monomers is about 1:6.5:2.5); AQUATERIC® (a mixture containing 66-73% of cellulose acetate phthalate (CAP), poloxamer and acetylated monoglycerides), AQUACOAT® CPD 30 (FMC) (30% by weight aqueous dispersion containing cellulose acetate phthalate (CAP) polymer); KOLLICOAT MAE® 3OD (ethyl acrylate/methacrylic acid copolymers wherein the ratio of free carboxyl groups to esters is about 1:1) 30DP (BASF) (methacrylic acid/ethyl acrylate copolymer, 30% dispersion); and EASTACRYL® 3OD (Eastman Chemical)((30% polymeric dispersion of methacrylic acid ethyl acrylate copolymer in water). In one embodiment, the enteric polymer is hydroxypropyl methylcellulose acetate succinate (HPMCAS).

The coating composition may further include pharmaceutically acceptable excipients, such as plasticizers, opacifiers and coloring agents. Examples of plasticizers include acetylated triacetin, triethyl citrate, tributyl citrate, glycerol tributyrate, diacetylated monoglyceride, polyethylene glycols, propylene glycol, sesame oil, acetyl tributyl citrate, acetyl triethyl citrate, diethyl oxalate, diethyl phthalate, diethyl maleate, diethyl fumarate, dibutyl succinate, diethylmalonate, dioctyl phthalate, dibutyl sebacate and mixtures of these. Examples of opacifiers include titanium dioxide, talc, calcium carbonate, behenic acid and cetyl alcohol. Examples of coloring agents include ferric oxide red, ferric oxide yellow, Lake of Tartrazine, Allura red, Lake of Quinoline yellow and Lake of Erythrosine.

In one embodiment, the formulation releases less than 50% of Nifurtimox after 8 hours. In another embodiment, the formulation releases less than 45% of Nifurtimox after 8 hours. In another embodiment, the formulation releases less than 40% of Nifurtimox after 8 hours.

In one embodiment, the formulation releases more than 70% of Nifurtimox after 24 hours. In another embodiment, the formulation releases more than 80% of Nifurtimox after 24 hours. In another embodiment, the formulation releases more than 90% of Nifurtimox after 24 hours.

The following examples are intended to be illustrative only, and not unduly limit the scope of the appended claims.

### Example 1

### Sustained Release Capsule Formulations

TABLE 1
Chemicals And Materials Used In The Development Of Sustained Release
Formulations Of Nifurtimox

| Chemical/material | Supplier |
|---|---|
| Avicel PH 101 | FMC Corp. (Newark, DE) |
| Avicel PH 102 | FMC Corp. (Newark, DE) |
| Eudragit ® NE 30D | Evonik Degussa Corp. (Piscataway, NJ) |
| Eudragit ® RS PO | Evonik Degussa Corp. (Piscataway, NJ) |
| Eudragit ® RL PO | Evonik Degussa Corp. (Piscataway, NJ) |
| Foremost #310 Regular | Foremost Harms (Baraboo, WI) |
| (Lactose Monohydrate) | |
| Hard Gelatin Capsules | Capsugel (Peapack, NJ) |
| (0SF White Opaque) | |
| Hydrochloric Acid | Mallinkrodt (St. Louis, MI) |
| Magnesium Stearate | Spectrum Chemicals (Gardena, CA) |
| Methocel K100M Premium CR | Colorcon Inc. (West Point, PA) |
| (IF10828) | |
| Methocel K100 Premium LV CR (IF 10807) | Colorcon Inc. (West Point, PA) |
| Methocel K100 M Premium (IF 10826) | Colorcon Inc. (West Point, PA) |
| Nifurtimox | WuXi AppTec Co., Ltd (Shanghai, China) |
| Phosphate Buffer Saline | EMD Chemicals (Gibbstown, NJ) |
| (10X PBS) | |
| Plasdone K29/32 | ISP Corp. (Wayne, New Jersey) |
| Polysorbate 80 (Tween 80) | Spectrum Chemicals (Gardena, CA) |
| Sodium Dodecyl Sulfate (SDS) | JT Baker (Phillipsburg, NJ) |
| Starch 1500 | Colorcon Inc. (West Point, PA) |

TABLE 2
Equipment Used In The Development of The Sustained
Released Formulation Of Nifurtimox

| Equipment | Manufacturer | Model |
|---|---|---|
| Capsule Filling Funnel | Torpac (Fairfield, NJ) | Size 0 Capsule |
| Dissolution Apparatus | Vanekl Industries (Edison, NJ) | VK7000 |
| Extruder | LCI Corporation (Charlette, NC) | MG-55 |
| KitchenAid Mixer | KitchenAid Inc (St. Joseph, MI) | K5SS |
| Planetary Mixer | Glascol LLC (Terre Haute, IN) | RD-20 |
| Marumerizer | LCI CORPORATION | QJ230T |
| (Spheronizer) | (CHARLOTTE, NC) | |
| Stainless Steel Sieves | Fisher Scientific (Pittsburgh, | Fisherbrand |
| | PA) | U.S. Standard |
| Tap Density Apparatus | Vankel Industries (Edison, NJ) | 10705 |

Extrusion-spheronization was used to develop matrix based Sustained Release formulations of Nifurtimox. A sustained release matrix formulation may comprise a drug, one or more water-swellable hydrophilic polymers, excipients such as fillers or binders, a flow aid (glident) and a lubricant. Other functional ingredients, such as buffering agents, stabilizers, solubilizers and surfactants, may also be included to improve or optimize the release performance of the formulation. Various water soluble, insoluble, and water swellable polymers, such as hydroxypropyl methylcellulose (HPMC), hydroxypropylcellulose (HPC), polyethylene oxide (PEO), and methacrylic copolymers have been used in bead form to achieve desired SR profiles. The mechanism of drug release from the beads depends on the type of the polymer used and the solubility of the active drug. In the case of hydrophilic polymers, drug release for soluble drugs occurs by diffusion of the drug through the hydrated portion of the matrix and erosion of the outer hydrated polymer on the surface of the matrix; for insoluble drugs, erosion is the predominant mechanism.

Examples of lubricants and glidents include stearic acid, magnesium stearate, calcium stearate, zinc stearate, sodium stearyl fumarate, glyceryl monostearate, glyceryl palmitostearate, hydrogenated vegetable oil, mineral oil, silicon dioxide, sodium lauryl sulfate, talc, sucrose esters of fatty acid, microcrystalline wax, yellow beeswax and white beeswax.

For the invention, Methocel (HPMC based), Eudragit® RL and Eudragit® RS (methacrylic copolymer based) were used to make beads with Sustained Release properties to fill into capsules. Initially several placebo formulations with different viscosity grades of methocel, percent of polymer, and amount of granulating liquid (deionized water) to make a wet mass were used to evaluate the extrusion-spheronization process. Based on Nifurtimox's solubility, dose requirement, and drug loading per capsule, five capsule formulations were optimized to make beads with SR properties.

### Example 2

### Preparation of Sustained Release Beads

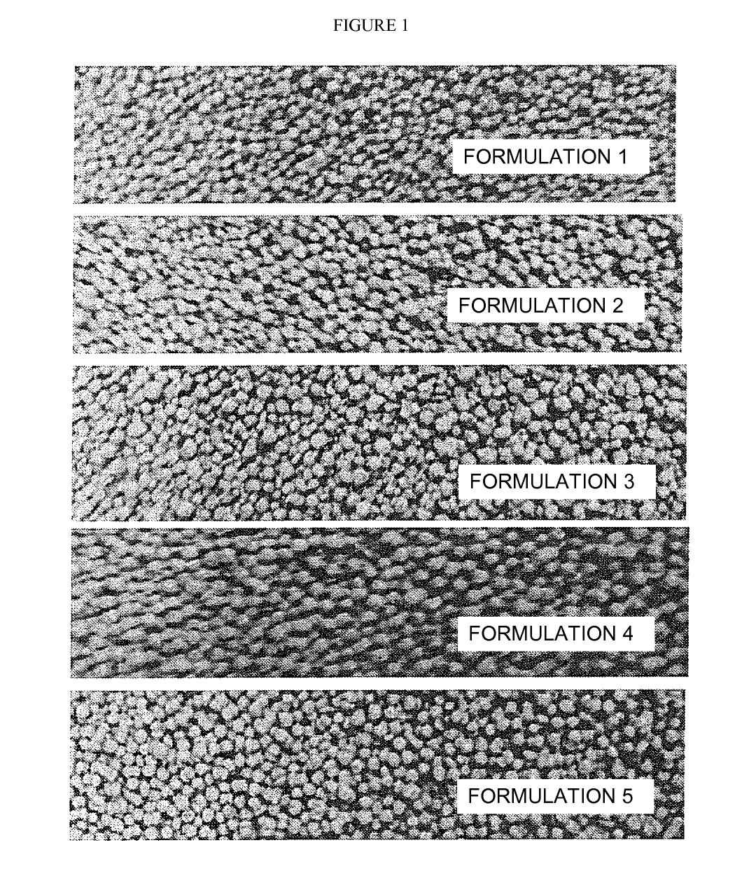

The total granulation batch size for each formulation listed in Table 3 was about 50 g. The dry excipients and active ingredients were blended in a Planetary Mixer (KitchenAid Inc., St. Joseph, Mich.) for ˜10 min. The blend was granulated in the same mixer using deionized water to achieve the appropriate consistency for extrusion. The granulations were extruded through a 1.0 mm×1.0 mm×22.6% (hole diameter×thickness×open area) stainless steel dome-discharge extrusion die using a lab-scale extruder (Model MG-55, LCI Corporation, Charlotte, N.C.). The extruded material was transferred to a spheronizer (Model QJ2 30T, LCI Corporation, Charlotte, N.C.) fitted with a 2.0 mm (space between grooves) crosshatched spheronizer plate (friction plate). Eudragit® polymer formulations were extruded at 30 rpm and spheronized at 1000 rpm for 30 sec. Formulations with Methocel were extruded at 30 rpm and spheronized at 1500 rpm for 3 min. The Eudragit® polymer beads were dried in a drier at 50° C. for 18 h, and the Methocel beads were dried at 50° C. for 2 h. FIG. 1 shows the Nifurtimox SR beads from different formulations.

TABLE 3
Formula for Preparation of Different Nifurtimox Capsule
Formulations

| | Formulation (% w/w/ on dry solids basis) | | | | | |
|---|---|---|---|---|---|---|
| | | | | | | Immediate |
| Formulation | Sustained Release (bead filled) | | | | | Release |
| Number | 1 | 2 | 3 | 4 | 5 | 6 |
| Ingredient | | | | | | |
| Nifurtimox | 20 | 35 | 35 | 35 | 35 | 35 |
| Avicel PH | 45 | 30 | 30 | 50 | 47.5 | 55 |
| 101 | | | | | | |
| Plasdone K | 5 | 5 | 5 | — | — | — |
| 29/32 | | | | | | |
| Eudragit ® | 30 | 30 | — | — | — | — |
| RS PO | | | | | | |
| Rudragit ® | — | — | 30 | — | — | — |
| RL PO | | | | | | |
| Lactose | — | — | — | 10 | 10 | 10 |
| Monohydrate | | | | | | |
| Methocel K | — | — | — | 5 | 7.5 | — |
| 100 | | | | | | |
| Premium LV | | | | | | |
| CR | | | | | | |
| Total (%) | 100.0 | 100.0 | 100.0 | 100.0 | 100.0 | 100.0 |
| Percent | 68.0 | 60.0 | 60.0 | 56.0 | 56.0 | — |
| Granulating | | | | | | |
| Liquid Used | | | | | | |
| (Deionized | | | | | | |
| Water) | | | | | | |

### Example 3

### Characterization of Beads

Beads of each formulation were sieved for 2 min by hand using nest of standard sieves size 12, 18, and 30 (1680, 1000, and 595 microns). The beads retained on each sieve were weighed and that data (Table 4) was used to construct percent bead size distribution curves. Bead formulations with Methocel (Formulations Nos. 4 and 5) had tighter size distribution and higher product yield compared to the bead formulations with Eudragit®. The size range of 1000 to 595 microns was considered appropriate, and the weight of beads in this range was reported as the product yield. For each formulation bead product yield, bulk and tapped density were determined, and Carr's index (%) and the Hausner ratio were calculated (Table 5). All SR bead formulations showed good flow properties based on the calculated Carr's index (<15) and Hausner ratio (<1.25).

TABLE 4
Sieve Analysis of Nifurtimox Sustained Release Bead Formulations

| | Bead Size (microns) | | | |
|---|---|---|---|---|
| | >1680 | 1000 | 595 | <595 |
| | Percent beads | Percent beads | Percent beads | Percent beads |
| Formulation | retained on Sieve | retained on Sieve | retained on Sieve | passed through |
| No. | 12 | 18 | 30 | Sieve 30 |
| 1 (SR) | 0 | 36.21 | 50.75 | 13.04 |
| 2 (SR) | 0 | 60.37 | 33.16 | 6.47 |
| 3 (SR) | 0 | 46.01 | 40.41 | 13.68 |
| 4 (SR) | 0 | 77.80 | 20.47 | 1.73 |
| 5 (SR) | 0 | 82.19 | 15.50 | 2.23 |

TABLE 5
Physical Characteristics of Nifurtimox Sustained Release Bead
Formulations

| | Total | Product | Bulk | Tapped | Carr's | |
|---|---|---|---|---|---|---|
| Formulation | Yield | yield | density | density | index | Hausner |
| No | (%) | (%) | (g/ml) | (g/ml) | (%) | ratio |
| 1 (SR) | 56.44 | 86.96 | 0.68 | 0.71 | 4.23 | 1.04 |
| 2 (SR) | 50.97 | 93.53 | 0.63 | 0.68 | 7.35 | 1.08 |
| 3 (SR) | 49.82 | 86.43 | 0.65 | 0.66 | 1.52 | 1.02 |
| 4 (SR) | 67.65 | 98.27 | 0.72 | 0.78 | 7.69 | 1.08 |
| 5 (SR) | 64.82 | 97.77 | 0.68 | 0.76 | 10.53 | 1.12 |

### Example 4

### Capsule Filling

About six capsules of each formulation were filled by hand with the help of a capsule-filling funnel (Torpac, Fairfield, N.J.) (FIG. 6) for size 0 capsules. Formulation 15825-41-1 was an immediate release powder formulation; all the other formulations were Sustained Release bead formulations. The particle size range for the bead formulations was 1000 to 595 microns. For each formulation, the average capsule fill weight and average Nifurtimox per capsule were calculated (Table 6).

TABLE 6
Average Capsule Fill Weight and Drug Per Capsule of
Nifurtimox Formulations
Capsule Size: 0

| | Average | | | Average |
|---|---|---|---|---|
| | empty | Average filled | | estimated |
| Formulation | capsule | capsule weight | Average fill | Nifurtimox per |
| No. | weight (g) | (g) | weight (g) | capsule (mg) |
| 1 (SR) | 0.095 | 0.596 | 0.501 | 100.11 |
| 2 (SR) | 0.095 | 0.524 | 0.430 | 150.34 |
| 3 (SR) | 0.095 | 0.525 | 0.430 | 150.67 |
| 4 (SR) | 0.095 | 0.523 | 0.429 | 150.02 |
| 5 (SR) | 0.096 | 0.524 | 0.428 | 149.95 |
| 6 (IR) | 0.095 | 0.380 | 0.285 | 99.65 |

### Example 5

### Capsule Content Assay

A. Capsule with Bead Formulation

The content of a capsule were emptied into a 100 ml volumetric flask using a weighing paper funnel. The capsule shell was tapped several times to remove all the beads. Dimethyl sulfoxide (DMSO, 10 ml) was added to the flask by volumetric pipette and the contents were sonicated for 10 min. if a visual inspection of the flask showed that beads were still present, then the flask was subjected to an additional 10 min of sonication. Once a suspension was achieved, the flask was filled to the 100 ml mark with acetonitrile. The flask was inverted and shaken multiple times to ensure proper mixing then left to stand for 10 min. A. 1.0 ml aliquot was taken with a volumetric pipette and transferred to a 10 ml volumetric flask. The flask was filled to the 10 ml mark with acetonitrile and mixed. A 1.5 ml aliquot was taken up in a syringe (all polypropylene); filtered through a 13 mm, 0.45 μm nylon membrane filter into a HPLC vial; and analyzed by high-performance liquid chromatography (HPLC).

B. Capsule with an Amorphous Powder Formulation

The contents of a capsule were emptied into a 100 ml volumetric flask fitted with a long step funnel. The capsule shell was rinsed with 3 ml of acetonitrile directly into the flask. The funnel was rinsed with acetonitrile (15 ml) and removed from the flask. The contents were sonicated for 10 min, then the flask was filled to the 100 mL mark with acetonitrile. The flask was inverted and shaken multiple times to ensure proper mixing, then left to stand for 10 min. A 1.0 ml aliquot was taken with a volumetric pipette and transferred to a 10 ml volumetric flask. The flask was filled to the mark with acetonitrile and mixed. A 1.5 ml aliquot was taken up in a syringe (all polypropylene); filtered through a 13 mm, 0.45 μm nylon membrane filter into a HPLC vial; and analyzed by HPLC.

Two capsules of each formulation were assayed; the expected and average Nifurtimox found per capsule are listed in Table 7. All HPLC findings were within the limits expected for the amount of Nifurtimox per capsule.

TABLE 7
Capsule Contents of Nifurtimox Formulations Analyzed by HPLC

| | Estimated | Average Nifurtimox | |
|---|---|---|---|
| | Nifurtimox | per capsule found by | Standard |
| Formulation No. | per capsule (mg) | HPLC (mg) | Deviation |
| 1 (SR) | 100.00 | 103.74 | 1.27 |
| 2 (SR) | 150.00 | 150.28 | 2.23 |
| 3 (SR) | 150.00 | 151.82 | 0.23 |
| 4 (SR) | 150.00 | 150.69 | 1.59 |
| 5 (SR) | 150.00 | 148.97 | 2.13 |
| 6 (IR) | 100.00 | 101.41 | 0.54 |

### Example 6

### Nifurtimox Solubility Studies

Nifurtimox is poorly soluble in water. To select a buffer for the in vitro release (dissolution) studies, solubility of Nifurtimox in possible buffers was determined by HPLC. For each 5 ml of the prepared buffer, drug was added in excess and mixed for 2 hr on a planetary mixer. Each sample was filtered through a syringe filter (0.45 μm nylon membrane) and analyzed by HPLC. Solubility results are listed in Table 8. Among the tested buffers, 0.1N HCl with 5% SDS had the highest solubility (308.46 μg/ml).

TABLE 8
Solubility of Nifurtimox

| Formulation | | Nifurtimox Found by HPLC |
|---|---|---|
| No. | Buffer | (μg/ml) |
| 7 | Water | 19.35 |
| 8 | 0.1N HCl | 90.61 |
| 9 | 0.1N HCl + 5% Tween 80 | 11.47 |
| 10 | 0.1N HCl + 5% SDS | 308.46 |
| 11 | 1X PBS | 13.06 |
| 12 | 1X PBS + 5% Tween 80 | 109.43 |
| 13 | 1X PBS + 5% SDS | 113.59 |

### Example 7

### In Vitro Release Studies

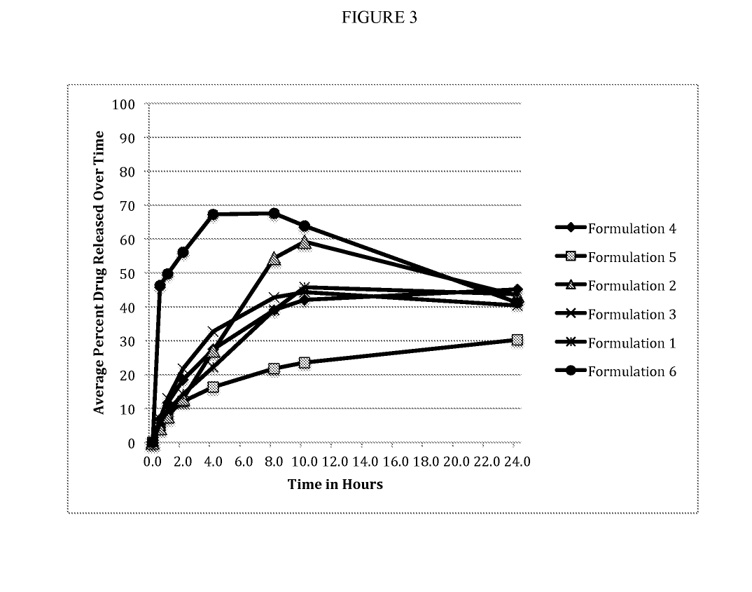

Nifurtimox release studies for each formulation were performed in duplicate using a standard USP dissolution apparatus II (Vankel, VK7000), at 100 rpm, in a medium of 900 mL 5% sodium dodecyl sulfate (SDS) in 0.1N hydrochloric acid (pH ˜1.2) at 37°±5° C. At specified time intervals (0.5, 1, 2, 4, 8, 10, and 24 h), 2 ml of sample was collected from each basket and replaced with the same volume of buffer. Collected samples were centrifuged for 10 min; 0.5 ml of sample was collected from the supernatant and analyzed by HPLC to determine the amount of Nifurtimox released at each time point. The average percent of Nifurtimox released over time was calculated (Table 9) and plotted (FIG. 3) to observe the SR achieved by each formulation.

TABLE 9
Average Percent Nifurtimox (in 0.1 N HCl with 5% SDS) Released Over Time

| | Formulation | | | | | | | | | | | |
|---|---|---|---|---|---|---|---|---|---|---|---|---|
| | Nifurtimox | | Nifurtimox | | Nifurtimox | | Nifurtimox | | Nifurtimox | | Nifurtimox | |
| | Capsule | | Capsule | | Capsule | | Capsule | | Capsule | | Capsule | |
| | Formulation | | Formulation | | Formulation | | Formulation | | Formulation | | Formulation | |
| | 4(SR) | | 5(SR) | | 2(SR) | | 3(SR) | | 1(SR) | | 6(IR)^{a} | |
| | Average | | Average | | Average | | Average | | Average | | Average | |
| | Percent | SD | Percent | | Percent | | Percent | | Percent | | Percent | |
| Time | Drug | (standard | Drug | | Drug | | Drug | | Drug | | Drug | |
| (hr) | Released | deviation) | Released | SD | Released | SD | Released | SD | Released | SD | Released | SD |
| 0 | 0 | 0 | 0 | 0 | 0 | 0 | 0 | 0 | 0 | 0 | 0 | N/A |
| 0.5 | 6.79 | 0.56 | 5.06 | 1.07 | 4.2 | 1.1 | 7.03 | 0.04 | 5.72 | 0.89 | 46.28 | N/A |
| 1 | 11.71 | 0.55 | 8.36 | 1.13 | 7.7 | 1.49 | 13.03 | 0.06 | 9.42 | 0.73 | 49.66 | N/A |
| 2 | 18.54 | 0.11 | 12.12 | 1.25 | 12.88 | 1.91 | 21.78 | 1.33 | 14.28 | 0.93 | 56.06 | N/A |
| 4 | 27.5 | 2.71 | 16.34 | 1.04 | 27.07 | 3.46 | 32.79 | 1.57 | 22.21 | 1.12 | 67.26 | N/A |
| 8 | 39.04 | 3.69 | 21.81 | 1.6 | 54.33 | 4.33 | 42.82 | 1.59 | 39.02 | 2.28 | 67.62 | N/A |
| 10 | 42.02 | 3.7 | 23.6 | 1.35 | 59.25 | 1.76 | 44.37 | 1.25 | 45.76 | 1.42 | 63.82 | N/A |
| 24 | 45.17 | 0.51 | 30.32 | 0.86 | 43.32 | 1.28 | 40.42 | 0.95 | 43.74 | 0.19 | 41.21 | N/A |

^{a}Formulation 6 was a direct powder filled, immediate release capsule formulation, and dissolution was not performed in duplicate.
N/A = Not Applicable.

During analysis of the dissolution samples (in 0.1N HCl with 5% SDS), about 25% of Nifurtimox degradation in 24 hr was observed and was evident from the low amounts of recovery at the 24 hr time point shown in FIG. 8. Further analysis of Nifurtimox degradation in dissolution samples is required to quantify the exact amount of Nifurtimox released over time.

Capsules formulations 2-5 contained 150 mg per capsule (size 0); capsules formulations 1 and 6 contained 100 mg of Nifurtimox per capsule. Maximum HPLC recovery observed was about 70% of the strength of the capsule; however, it was difficult to estimate the exact amount of degradation of Nifurtimox over time before the capsules were analyzed. Without considering the amount of Nifurtimox degraded over time, the percent of drug release observed in the SR formulations ranged from 23.6% to 59.25% in 10 hr, whereas IR formulation released about 63.92%.

### Example 8

### Nifurtimox Stability in 2% W/V SDS in Deionized Water

Due to the degradation of Nifurtimox observed in dissolution buffer (0.1 N HCl with 5% SDS), a preliminary stability study of Nifurtimox in 2% w/v SDS in deionized water was performed. To 20 ml of 2% SDS solution, an excess amount of Nifurtimox was added and the solution was stirred with a magnetic stirrer for 45 min. The solution was filtered using a 0.45 μm nylon syringe filter, and aliquots of 1 ml samples were collected in HPLC vials and stored at different temperatures (25° C. and 37° C.) for different time periods (0 to 24 hr). HPLC analysis results are presented in Table 10. Based on the amount of Nifurtimox found in initial (0 hr) and final (24 h) samples, there is no significant degradation of Nifurtimox in 2% w/v SDS (in water) at 25° C. and 37° C. for up to 24 hr.

TABLE 10
Nifurtimox Stability in 2% W/V SDS (In Water)

| | | | | Estimated |
|---|---|---|---|---|
| | | | | Amount of |
| | Temp | Time | Peak Area | Nifurtimox |
| Sample Number | (° C.) | (hr) | (9.6 min) | (μg/ml) |
| 1 | 25 | 0 | 13727.20 | 344.22 |
| 2 | | 4 | 13558.00 | 339.96 |
| 3 | | 8 | 13773.00 | 345.38 |
| 4 | | 12 | 13806.00 | 346.21 |
| 5 | | 16 | 13835.00 | 346.94 |
| 6 | | 20 | 13755.00 | 344.92 |
| 7 | | 24 | 13631.00 | 341.80 |
| Control 1 | 25 | 0 | 0.00 | 0.00 |
| Control 2 | | | 0.00 | 0.00 |
| Control 3 | | | 0.00 | 0.00 |
| Control 4 | | | 0.00 | 0.00 |
| 8 | 37 | 1 | 13511.00 | 338.78 |
| 9 | | 2 | 13549.00 | 339.74 |
| 10 | | 3 | 13511.00 | 338.78 |
| 11 | | 24 | 13709.00 | 343.76 |
| 12 | | 24 | 13542.00 | 339.56 |

### Example 9

### In Vitro Release Studies Using 2% W/V SDS in Deionized Water as Dissolution Buffer

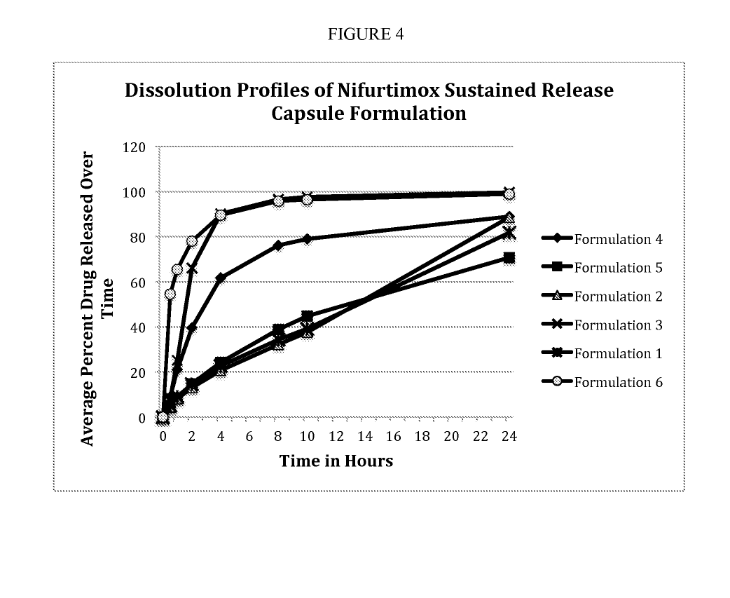

As described in earlier sections, the Nifurtimox release (dissolution) studies were repeated using a dissolution buffer of 2% w/v SDS in deionized water. Dissolution of each formulation was performed in duplicate. The average percent of Nifurtimox released over time was calculated (Table 11) and plotted (FIG. 4) to observe the SR achieved by each formulation.

TABLE 11?
Average Percent Nifurtimox (in 2% WN SDS in Water) Released Over Time

| | Formulation | | | | | | | | | | | |
|---|---|---|---|---|---|---|---|---|---|---|---|---|
| | Nifurtimox | | Nifurtimox | | Nifurtimox | | Nifurtimox | | Nifurtimox | | Nifurtimox | |
| | Capsule | | Capsule | | Capsule | | Capsule | | Capsule | | Capsule | |
| | Formulation | | Formulation | | Formulation | | Formulation | | Formulation | | Formulation | |
| | 4(SR) | SD | 5(SR) | | 2(SR) | | 3(SR) | | 1(SR) | | 6(IR)^{a} | |
| | Average | (stan- | Average | | Average | | Average | | Average | | Average | |
| | Percent | dard | Percent | | Percent | | Percent | | Percent | | Percent | |
| Time | Drug | devia- | Drug | | Drug | | Drug | | Drug | | Drug | |
| (hr) | Released | tion) | Released | SD | Released | SD | Released | SD | Released | SD | Released | SD |
| 0 | 0.00 | 0.00 | 0.00 | 0.00 | 0.00 | 0.00 | 0.00 | 0.00 | 0.00 | 0.00 | 0.00 | 0.00 |
| 0.5 | 9.05 | 0.50 | 4.37 | 0.58 | 4.65 | 0.49 | 8.48 | 0.53 | 4.95 | 0.45 | 54.56 | 1.51 |
| 1 | 20.98 | 2.07 | 8.53 | 0.77 | 8.16 | 0.68 | 25.22 | 1.03 | 8.97 | 0.21 | 65.40 | 0.87 |
| 2 | 39.69 | 3.46 | 14.73 | 1.40 | 13.12 | 0.53 | 66.05 | 1.66 | 14.64 | 0.35 | 78.00 | 1.17 |
| 4 | 61.70 | 1.36 | 24.26 | 0.70 | 20.73 | 1.40 | 90.09 | 0.11 | 22.67 | 0.15 | 89.54 | 2.20 |
| 8 | 76.12 | 2.73 | 38.93 | 1.42 | 32.18 | 1.08 | 96.56 | 0.96 | 34.35 | 0.88 | 95.68 | 2.50 |
| 10 | 79.02 | 3.17 | 44.85 | 1.48 | 37.69 | 1.12 | 97.64 | 0.50 | 39.21 | 0.72 | 96.41 | 2.99 |
| 24 | 88.90 | 3.84 | 70.75 | 2.94 | 88.62 | 2.19 | 99.71 | 1.16 | 81.78 | 2.90 | 98.90 | 2.83 |

^{a}Formulation 6 was a direct powder filled, immediate release capsule formulation, and dissolution was not performed in duplicate.

HPLC analysis determined the maximum recovery was about 98.90% of the strength of the capsule (formulation 6) and no significant instability of Nifurtimox was observed with the dissolution performed in 2% w/v SDS in deionized water. Among the tested SR formulations, the percent Nifurtimox release observed ranged from 37.69% to 97.64% in 10 hr, whereas the IR formulation released about 96.4%. More controlled release was observed as the percentage of Methocel polymer was increased from 5% to 7.5% in the formulation (formulations 4 and 5; FIG. 4). Formulation 2 with Eudragit® RS PO achieved higher controlled release compared to formulation 3 with Eudragit® RL PO (FIG. 4).

From the results shown above, sustained release capsule formulations of Nifurtimox were formulated by extrusion spheronization using Methocel and Eudragit® polymers. SR capsule formulations of Nifurtimox (150 mg) prepared using 5% Methocel (Formulation 4) resulted in sustained drug release of ˜79% in 10 hr and ˜89% within 24 hr and with 7.5% Methocel (Formulation 5) the sustained release observed was ˜44% in 10 hr and ˜70% within 24 hr. Nifurtimox capsule formulations (150 mg) prepared using Eudragit® RS PO (Formulation 2) achieved a sustained release of ˜37% in 10 hr and ˜88% within 24 hr compared to Eudragit® RL PO (Formulation 3) which resulted in ˜90% drug release within 4 hr.

The results demonstrated above show that the formulations of the present invention has significantly improved sustained-release profile for treating the Chagas disease and other diseases or disorders that are treatable by Nifurtimox. This not only reduces the toxicities associated with immediate-release Nifurtimox, but also improves patient compliance for less frequent administration. Therefore, it is expected that the formulations of this invention to have wide applications on different diseases that are treatable by Nifurtimox.

What is claimed is:

1. A formulation for controlled release of Nifurtimox, comprising:
a) a therapeutically effective amount of Nifurtimox;
b) a water-swellable hydrophilic polymer; and
c) a binder;
wherein the formulation continuously releases Nifurtimox for at least 24 hours.

2. The formulation of claim 1, wherein the formulation is in multi-particulate form enclosed in a capsule.

3. The formulation of claim 2, wherein the capsule comprises enteric materials.

4. The formulation of claim 2, wherein the particulates in the multi-particulate formulation is further coated by an enteric material.

5. The formulation of claim 4, wherein the enteric material is selected from the group consisting of: hydroxypropyl methylcellulose acetate succinate (HPMCAS), hydroxypropyl methylcellulose phthalate (HPMCP), polyvinyl acetate phthalate, cellulose acetate phthalate, cellulose acetate trimellitate, shellac, zein, polymethacrylates containing carboxyl groups, amylose acetate phthalate, styrene maleic acid copolymer, cellulose acetate succinate, Poly(methacylic acid-co-methyl methacrylate) 1:1, Poly(methacylic acid-co-methyl methacrylate) 1:2, EUDRAGIT® L, Poly(methacrylic acid-co-ethyl acrylate) 1:1, Poly(methacylic acid-co-ethyl acrylate) 1:1, Poly(methyl acrylate-co-methyl methacrylate-co-methacrylic acid) 7:3:1; cellulose acetate hydrogen 1,2-benzenedicarboxylate, AQUACOAT® CPD 30, poly(vinyl acetate) dispersion 30 percent, and EASTACRYL® 3OD.

6. The formulation of claim 1, wherein said water-swellable hydrophilic polymer is selected from the group consisting of: polyvinylpyrrolidone, poly(ethyl acrylate-co-methyl methacrylate-co-trimethylammonioethyl methacrylate chloride) 1:2:0.1, poly(ethyl acrylate-co-methyl methacrylate-co-trimethylammonioethyl methacrylate chloride) 1:2:0.2, hydroxypropyl methylcellulose, and combinations thereof.

7. The formulation of claim 1, wherein said formulation comprises 10 to 40% w/w on dry solids basis of Nifurtimox.

8. The formulation of claim 7, wherein said binder is microcrystalline cellulose, polyvinylpyrrolidone, lactose monohydrate, or combinations thereof.

9. The formulation of claim 7, wherein said binder is microcrystalline cellulose, and said water-swellable hydrophilic polymer is poly(ethyl acrylate-co-methyl methacrylate-co-trimethylammonioethyl methacrylate chloride) 1:2:0.1, poly(ethyl acrylate-co-methyl methacrylate-co-trimethylammonioethyl methacrylate chloride) 1:2:0.2, hydroxypropyl methylcellulose, and combinations thereof.

10. The formulation of claim 8, wherein said formulation comprises 20 to 60% w/w on dry solids basis of microcrystalline cellulose.

11. The formulation of claim 8, wherein said formulation comprises 20 to 40% w/w on dry solids basis of poly(ethyl acrylate-co-methyl methacrylate-co-trimethylammonio ethyl methacrylate chloride) 1:2:0.1.

12. The formulation of claim 8, wherein said formulation comprises 1 to 10% w/w on dry solids basis of polyvinylpyrrolidone.

13. The formulation of claim 8, wherein said formulation comprises 1 to 10% w/w on dry solids basis of hydroxypropyl methylcellulose.

14. The formulation of claim 1, wherein said formulation releases less than 50% of Nifurtimox after 8 hours.

15. (canceled)

16. A formulation for controlled release of Nifurtimox, comprising:
a) 10 to 40% w/w on dry solids basis of Nifurtimox;
b) 20 to 40% w/w on dry solids basis of at least one water-swellable hydrophilic polymer; and
c) 20 to 60% w/w on dry solids basis of a binder;
wherein said at least one water-swellable hydrophilic polymer is selected from the group consisting of: poly(ethyl acrylate-co-methyl methacrylate-co-trimethylammonioethyl methacrylate chloride) 1:2:0.1, poly(ethyl acrylate-co-methyl methacrylate-co-trimethylammonioethyl methacrylate chloride) 1:2:0.2, hydroxypropyl methylcellulose, and combinations thereof;
wherein said binder is microcrystalline cellulose, polyvinylpyrrolidone, lactose monohydrate, or combinations thereof; and
wherein the formulation continuously releases Nifurtimox for at least 12 hours.

17. A method of treating a patient having Chagas disease, comprising the steps of:
a) administering, once daily, to the patient a sustained release capsule formulation of Nifurtimox;
wherein said sustained release capsule formulation of Nifurtimox comprises: (1) a therapeutically effective amount of Nifurtimox, (b) a water-swellable hydrophilic polymer, and (c) a binder; and
wherein said sustained release capsule formulation of Nifurtimox continuously releases Nifurtimox for at least 24 hours.

18. The method of claim 17, wherein said binder is microcrystalline cellulose and said water-swellable hydrophilic polymer is poly(ethyl acrylate-co-methyl methacrylate-co-trimethylammonioethyl methacrylate chloride) 1:2:0.1, poly(ethyl acrylate-co-methyl methacrylate-co-trimethylammonioethyl methacrylate chloride) 1:2:0.2, hydroxypropyl methylcellulose, and combinations thereof.

19. The method of claim 18, wherein said formulation comprises 20 to 60% w/w on dry solids basis of microcrystalline cellulose.

20. The method of claim 18, wherein said formulation comprises 1 to 10% w/w on dry solids basis of polyvinylpyrrolidone.

21. The method of claim 18, wherein said formulation comprises 20 to 40% w/w on dry solids basis of poly(ethyl acrylate-co-methyl methacrylate-co-trimethylammonioethyl methacrylate chloride) 1:2:0.1.

22. The method of claim 18, wherein said formulation comprises 1 to 10% w/w on dry solids basis of hydroxypropyl methylcellulose.

23. The method of claim 17, wherein said formulation releases less than 50% of Nifurtimox after 8 hours.

24. (canceled)
